(19) United States
(12) Patent Application Publication
(10) Pub. No.: US 2026/0315221 A1
(43) Pub. Date: Oct. 15, 2026

(54) HYBRID MATERIAL STENT FOR TRANSCATHETER AORTIC VALVE REPLACEMENT

(71) Applicant: St. Jude Medical, Cardiology Division, Inc., St. Paul, MN (US)

(72) Inventors: Heath Marnach, Minneapolis, MN (US); William H. Peckels, Robbinsdale, MN (US); Prem Midha, St. Paul, MN (US)

(73) Assignee: St. Jude Medical, Cardiology Division, Inc., St. Paul, MN (US)

(21) Appl. No.: 19/482,131

(22) PCT Filed: Apr. 23, 2024
(86) PCT No.: PCT/US2024/025775

§ 371 Date: Nov. 6, 2025

Related U.S. Application Data
(60) Provisional application No. 63/503,001, filed on May 18, 2023.

(51) Int. Cl. A61F 2/24

(52) U.S. Cl. CPC A61F 2/2418; A61F 2220/0033; A61F 2220/0075; A61F 2230/0006; A61F 2230/0017; A61F 2230/0086; A61F 2250/0037

(57) ABSTRACT

A prosthetic heart valve may include a balloon-expandable frame formed of a plastically-expandable material, and a self-expanding frame formed of a self-expanding material, the self-expanding frame including a plurality of commissure attachment features. The valve may include a plurality of prosthetic leaflets, wherein pairs of adjacent prosthetic leaflets form commissures, each of the commissures being coupled to a corresponding one of the plurality of commissure attachment features. The balloon-expandable frame may be coupled to the self-expanding frame to form a hybrid frame, the hybrid frame including an inflow end and an outflow end, the balloon-expandable frame being positioned at the inflow end of the hybrid frame, and the self-expanding frame being positioned at the outflow end of the hybrid frame.

### CROSS-REFERENCE TO RELATED APPLICATIONS

This application claims the benefit of priority of the filing date of U.S. Provisional Patent Application No. 63/503,001, filed May 18, 2023, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves, including surgical heart valves and collapsible/expandable heart valves intended for transcatheter aortic valve replacement or implantation (“TAVR” or “TAVI”) or transcatheter mitral valve replacement (“TMVR”), are well known in the patent literature. Surgical or mechanical heart valves may be sutured into a native annulus of a patient during an open-heart surgical procedure, for example. Collapsible/expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus.

Collapsible/expandable prosthetic heart valves typically take the form of a one-way valve structure (often referred to as a valve assembly) mounted to/within an expandable stent (the terms “stent” and “frame” are used interchangeably herein). In general, these collapsible/expandable heart valves include a self-expanding or balloon-expandable stent, often made of nitinol or another shape-memory metal or metal alloy (for self-expanding stents) or steel or cobalt chromium (for balloon-expandable stents). Existing collapsible/expandable TAVR devices have been known to use different configurations of stent layouts-including straight vertical struts connected by “V”s as illustrated in U.S. Pat. No. 8,454,685, or diamond-shaped cell layouts as illustrated in U.S. Pat. No. 9,326,856, both of which are hereby incorporated herein by reference. The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon-expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

Self-expanding and balloon-expandable stent frames typically each have certain benefits as well as certain drawbacks, such that a tradeoff is typically required when either a self-expanding stent frame is used for a prosthetic heart valve, or when a balloon-expandable stent frame is used for a prosthetic heart valve. It would be desirable to achieve a stent-frame that can be used in a prosthetic heart valve that maximizes the benefits and/or minimizes the drawbacks of either particular style of stent frame.

### SUMMARY OF THE DISCLOSURE

According to one aspect of the disclosure, a prosthetic heart valve includes a balloon-expandable frame formed of a plastically-expandable material, and a self-expanding frame formed of a self-expanding material, the self-expanding frame including a plurality of commissure attachment features. The prosthetic heart valve may include a plurality of prosthetic leaflets, wherein pairs of adjacent prosthetic leaflets form commissures, each of the commissures being coupled to a corresponding one of the plurality of commissure attachment features. The balloon-expandable frame may be coupled to the self-expanding frame to form a hybrid frame, the hybrid frame including an inflow end and an outflow end, the balloon-expandable frame being positioned at the inflow end of the hybrid frame, and the self-expanding frame being positioned at the outflow end of the hybrid frame. The plurality of prosthetic leaflets may be positioned within the hybrid frame along an axial extent that spans both the self-expanding frame and the balloon-expandable frame. The balloon-expandable frame may include a first inflow-most row of cells, and a second row of cells positioned downstream of the first row of cells, and the self-expanding frame may include a third row of cells. The balloon-expandable frame may be coupled to the self-expanding frame via connections between the second row of cells and the third row of cells. The connections may each include one of a protrusion or recess positioned at an outflow apex of a cell in the second row of cells, and the other of a protrusion or recess positioned at an inflow apex of a cell in the third row of cells, the protrusion being received within the recess. A buffer material may be positioned between each corresponding protrusion and recess to prevent metal-on-metal contact at the connections. Each protrusion or recess may be wedged in a thickness direction of the hybrid frame to prevent the self-expanding frame from sliding radially inwardly with respect to the balloon-expandable frame at the connections. The protrusion and the recess may form a dovetail connection. One or more sutures may wrap around each of the connections to secure the protrusion within the recess. An outflow apex of a cell in the second row of cells may be positioned between two circumferentially adjacent inflow apices of two adjacent cells in the third row of cells. The connections may each include one of a protrusion or recess positioned on a strut of the cell in the second row of cells, and the other of a protrusion or recess positioned on a strut of one of the two circumferentially adjacent cells in the third row of cells, the protrusion being received within the recess.

According to another aspect of the disclosure, a prosthetic heart valve includes a balloon-expandable frame formed of a plastically-expandable material, and a self-expanding frame formed of a self-expanding material, the self-expanding frame including a plurality of commissure attachment features. The prosthetic heart valve may include a plurality of prosthetic leaflets, wherein pairs of adjacent prosthetic leaflets form commissures, each of the commissures being coupled to a corresponding one of the plurality of commissure attachment features. The self-expandable frame may be coupled to, and positioned radially within, the balloon-expandable frame to form a hybrid frame. In an expanded condition of the hybrid frame, the balloon-expandable frame may have a first axial height and the self-expanding frame may have a second axial height about equal to the first axial height. The balloon-expandable frame may include a plurality of circumferential rows of diamond-shaped cells, and the self-expanding frame may include a plurality of rows of diamond-shaped cells. The balloon-expandable frame may include fewer circumferential rows of diamond-shaped cells than the self-expanding frame. Each diamond-shaped cell of the balloon-expandable frame may define a first area that is larger than an area of any diamond-shaped cell of the self-expanding frame.

According to still another aspect of the disclosure, a prosthetic heart valve includes a balloon-expandable frame formed of a plastically-expandable material, the balloon-expandable frame including a plurality of commissure attachment features. The prosthetic heart valve may include a self-expanding frame formed of a self-expanding material, and a plurality of prosthetic leaflets, wherein pairs of adjacent prosthetic leaflets form commissures, each of the commissures being coupled to a corresponding one of the plurality of commissure attachment features. The self-expanding frame may be coupled to the plurality of commissure attachment features to form a hybrid frame, the self-expanding frame being positioned radially outward of the balloon-expandable frame at an axial height that aligns with the plurality of commissure attachment features. Each of the plurality of commissure attachment features may be cantilevered with respect to the balloon-expandable frame. The balloon-expandable frame may include two circumferential rows of diamond-shaped cells adjacent to an inflow end of the hybrid frame, and the self-expanding frame may include one circumferential row of diamond-shaped cells adjacent to an outflow end of the hybrid frame. In the absence of applied forces, the self-expanding frame may be either (i) circular; or (ii) lobed, with one lobe positioned circumferentially between each pair of circumferentially adjacent commissure attachment features, each lobe extending a first distance from a radial center of the self-expanding frame, the self-expanding frame having connection locations where the self-expanding frame is coupled to the plurality of commissure attachment features, each connection location extending a second radial distance from the radial center of the self-expanding frame, the first distance being greater than the second distance.

### BRIEF DESCRIPTION OF DRAWINGS

FIG. 1 is a front view of a self-expandable stent frame, in an expanded condition, for use in a prosthetic heart valve.

FIGS. 2-3 are front and top perspective views, respectively, of a balloon expandable stent frame, in an expanded condition, for use in a prosthetic heart valve.

FIG. 4 is a perspective view of a prosthetic heart valve with a hybrid frame.

FIG. 5 is an enlarged front view of a portion of the hybrid frame of FIG. 4 where two frame portions connect.

FIG. 6 is an enlarged perspective view of the hybrid frame of FIG. 5 where two frame portions connect.

FIG. 7 is a side view of a hybrid frame for a prosthetic heart valve according to another aspect of the disclosure.

FIG. 8 is an enlarged side view of the hybrid frame of FIG. 7 where two frame portions connect.

FIGS. 9-10 are views, from different angles, of a hybrid frame for a prosthetic heart valve according to a further aspect of the disclosure.

FIGS. 11-14 are enlarged isolated views of alternative connection options to connect a first frame portion to a second frame portion.

FIG. 15 is a perspective view of a hybrid frame of a prosthetic heart valve according to another aspect of the disclosure.

FIG. 16 illustrates an alternate shape for the commissure ring of FIG. 15.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta when the prosthetic heart valve is implanted as intended. The intended position and orientation are used for the convenience of describing the valve disclosed herein, however, it should be noted that the use of the valve is not limited to the intended position and orientation but may be deployed in any type of lumen or passageway. For example, although the prosthetic heart valve is described herein as a prosthetic aortic valve, the same or similar structures and features can be employed in other heart valves, such as the pulmonary valve, the mitral valve, or the tricuspid valve. As used herein, the terms “substantially,” “generally,” “approximately,” and “about” are intended to mean that slight deviations from absolute are included within the scope of the term so modified. As used herein, the stent may assume an “expanded state” and a “collapsed state,” which refer to the relative radial size of the stent.

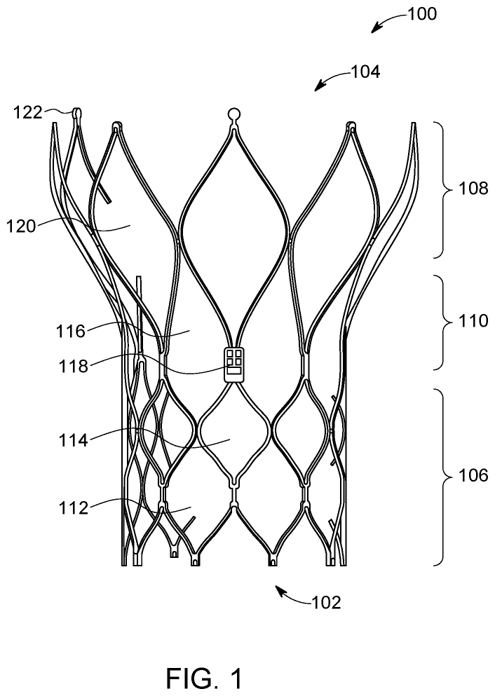

FIG. 1 illustrates a self-expanding frame 100 that may be used, for example, as part of a prosthetic aortic valve. It should be understood that, in FIG. 1, rear portions of the frame 100 are omitted for purposes of clarity. Frame 100 may extend from an inflow end 102 to an outflow end 104. Generally, frame 100 may include a generally cylindrical annulus section 106 nearer the inflow end 102, an outwardly flared (e.g. bell-shape) aortic section 108 nearer the outflow end 104, and a transition section 110 between the annulus section 106 and the aortic section 108.

Frame 100 may be formed of as a monolithic unit from a material having shape memory properties, such as nickel titanium alloys, including Nitinol. Frame 100 may be formed by laser cutting a tube of Nitinol, and then setting the desired expanded shape (such as that shown in FIG. 1) via heat treatment. In the illustrated embodiment, the annulus section 106 includes two circumferential rows of generally diamond-shaped cells, including a first inflow-most row of cells 112, and a second adjacent row of cells 114. It should be understood that more or fewer rows of cells may be provided, with more or fewer cells per row than shown, and the cells may have other shapes besides a generally diamond-shape. Although not shown, an inner cuff may be provided on the interior of the frame 100 along part or all of the annulus section 106, and an outer cuff may be provided on the exterior of the frame 100 along part of all of the annulus section 106. It should be understood that an inner cuff may be provided without an outer cuff, and outer cuff may be provided without an inner cuff, or both cuffs may be provided simultaneously. If one or more cuffs are provided, they may be formed of biological materials (e.g. bioprosthetic tissue such as porcine or bovine pericardium), synthetic materials (e.g. PET, PTFE, UHMWPE, etc.), or combinations thereof.

In the illustrated embodiment of frame 100, a single row of cells 116 help transition between the relatively small annulus section 106 to the relatively large aortic section 108, such that at least parts of the cells 116 (which may be generally diamond-shaped) extend radially outwardly from a central longitudinal axis of the frame 100 when the frame 100 is in the expanded condition. The frame may define a plurality of commissure attachment features (“CAFs”) 118 that are to help secure prosthetic leaflets to the frame 100. Although not shown, the prosthetic heart valve that incorporates frame 100 also includes one or more prosthetic leaflets, which may coapt with each other to prevent blood from flowing from the outflow end 104 to the inflow end 102, and move away from each other to allow blood to flow from the inflow end 102 to the outflow end 104, as the heart beats. The prosthetic leaflets may be formed of materials described above in connection with the cuffs. In the illustrated embodiment, three CAFs 118 are provided and three prosthetic leaflets would be provided, with each adjacent par of prosthetic leaflets being connected to each other and the frame 100 via the CAFs 118. In the illustrated embodiment, the CAFs 118 are formed as generally rectangular members, formed integrally with the frame 100, and including a plurality of apertures to assist with sutures passing through the prosthetic leaflets and the CAFs 118 to connect the components. However, it should be understood that other types of CAFs 118, including CAFs having different shapes, different numbers, and/or different positions, may be used in place of CAFs 118. In some prosthetic heart valves, the frame does not include any specific structure for use as a CAF, but rather the struts that form the cells may be used as points of connection to the prosthetic leaflets, effectively acting as CAFs.

Still referring to FIG. 1, the frame 100 includes a row of cells 120 at the outflow end 104 of the frame 100 in the aortic section 108, with the cells 120 being generally diamond-shaped. In the illustrated embodiment, cells 120 are relatively large compared to cells 112, 114, and preferably remain uncovered by any cuff or leaflet so that blood can flow through the large cells 120 without obstructing access to the coronary arteries, which begin just downstream of the native aortic valve. The cells 120, when the frame 100 is expanded, extend farther radially outwardly form the central longitudinal axis of the frame 100 so that the aortic section 108 defines the largest diameter of the frame 100.

In use, the prosthetic heart valve incorporating the frame 100 is typically maintained collapsed in a delivery condition within a sheath of a catheter as it is delivered intravascularly (e.g. through the femoral artery, around the aortic arch, and into the native aortic valve). To deploy the prosthetic heart valve, the overlying sheath may be withdrawn, allowing the prosthetic heart valve, via frame 100, to self-expand. One or more of the cells 120 in the aortic section 108 may include retainers 122 extending in the outflow direction from outflow apexes of cells 120. These retainers 122 may interact with complementary features of a delivery device (not shown) to help maintain the frame 100 connected to the delivery device prior to full expansion and release (e.g. prior to the overlying sheath fully uncovering the frame 100). Retainers 122 may be helpful because frame 100 tends to expand to the illustrated shape in the absence of applied forces, and thus maintaining a connection can help prevent premature release and full deployment of the frame 100 during use.

As noted above, frames formed of materials that are suitable for self-expandable frames, such as Nitinol, may have both benefits and drawbacks over other materials that are typically used in balloon-expandable frames (which may also be referred to as plastically expandable frames, and which are described in greater detail below). After full deployment and expansion of the prosthetic heart valve that incorporates frame 100, the prosthetic heart valve is maintained in place largely due to the expansion forces of the frame 100 contacting tissue at or around the native aortic valve. However, materials used in self-expanding frames, including Nitinol, typically have low stiffness. As a result of this low stiffness, the anchoring force purely from radially-outward expansion forces of frame 100 are relatively small. Thus, the outwardly-flared aortic section 108 (which may include the outwardly flared portions of the transition section 110) help to provide additional anchoring, particularly against migration of the frame 100 into the left ventricle when the prosthetic leaflets are closed. In other words, the diameter of the aortic section 108, when expanded, is larger than the inner diameter of the native aortic valve, and thus the aortic section 108 helps to provide additional anchoring force to supplement the radial anchoring force. The requirement of this outwardly flared section of frame 100 inherently requires the frame 100 to extend a longer length in the outflow direction compared to a frame that could fully omit the aortic section 108. As noted above, the coronary arteries begin just beyond the native aortic valve in the outflow direction. The additional material of frame 100 at the aortic section 108 may thus be detrimental because (i) blood flow into the coronary arteries may become obstructed and/or (ii) future access to the coronary arteries (e.g. in order to deploy a coronary stent within the coronary arteries) may become difficult or impossible.

Although the low stiffness of Nitinol and other similar materials used to form self-expanding frames may be a drawback because it may require additional length to achieve suitable anchoring, the low stiffness may also provide certain benefits. When the prosthetic leaflets coapt to prevent retrograde blood flow from the aorta back to the left ventricle, stress is created at the point of attachment of the prosthetic leaflets to the frame 100 at the CAFs 118. The lower stiffness of the material of frame 100 allows the frame 100 to deflect inwardly at and/or near the CAFs 118 when the prosthetic leaflets coapt. This inward deflection helps to better distribute the forces applied at the leaflet-CAF connection, meaning that less stress is applied to the soft tissue (or synthetic/fabric) leaflets. This, in turn, may lead to a greater durability of the prosthetic leaflets over time, as the applied stress to the leaflets during the normal operation of the prosthetic heart valve is reduced. Stated more simply, the material of self-expanding frames enhances durability of the “soft” components of the prosthetic heart valve (a benefit) but typically requires increased length of the frame extending into the aorta (a drawback).

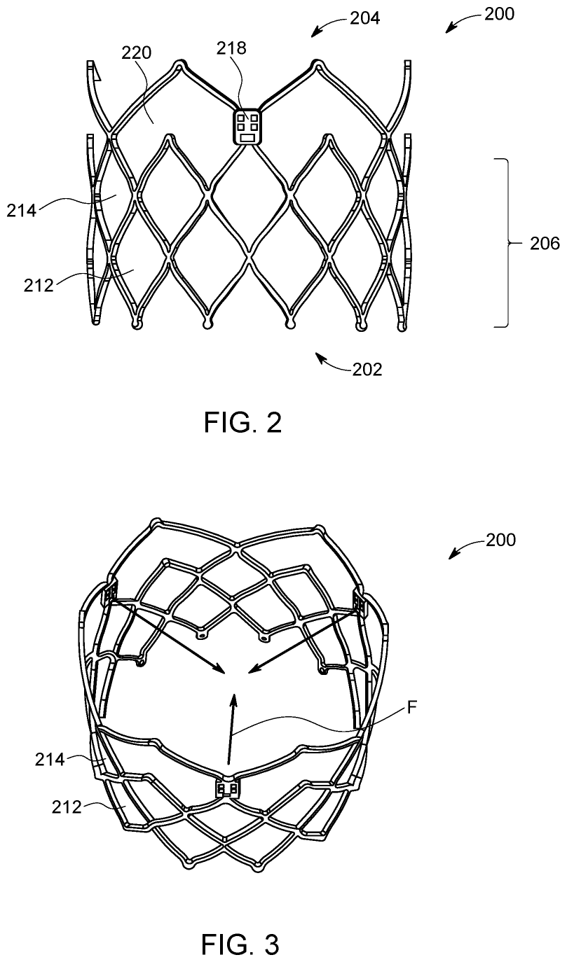

FIGS. 2-3 illustrate a balloon-expandable (or plastically expandable) frame 200 that may be used, for example, as part of a prosthetic aortic valve. It should be understood that, in FIG. 2, rear portions of the frame 200 are omitted for purposes of clarity. Frame 200 may extend from an inflow end 202 to an outflow end 204. Frame 200 may be generally cylindrical and include an annulus section 206.

Frame 200 may be formed of as a monolithic unit from a material having properties suitable for plastic expansion, such as stainless steel or cobalt chromium. Frame 200 may be formed by laser cutting a tube of the specific material. In the illustrated embodiment, the annulus section 206 includes two circumferential rows of generally diamond-shaped cells, including a first inflow-most row of cells 212, and a second adjacent row of cells 214. It should be understood that more or fewer rows of cells may be provided, with more or fewer cells per row than shown, and the cells may have other shapes besides a generally diamond-shape. Although not shown, an inner cuff may be provided on the interior of the frame 200 along part or all of the annulus section 206, and an outer cuff may be provided on the exterior of the frame 200 along part of all of the annulus section 206. It should be understood that an inner cuff may be provided without an outer cuff, and outer cuff may be provided without an inner cuff, or both cuffs may be provided simultaneously. If one or more cuffs are provided, they may be formed of any of the materials described above in connection with frame 100.

In the illustrated embodiment of frame 200, a single row of outflow cells 220 are positioned downstream the first two rows 212, 214. Outflow cells 220 may be irregularly shaped and define a significantly larger area than defined by cells in the first two rows 212, 214. Similar to frame 100, frame 200 may define a plurality of CAFs 218 that may be similar or identical in number, position, size, etc. to CAFs 118. As with CAFs 118, CAFs 218 may be provided in different numbers, positions, and/or shapes than shown, with three being included in the embodiment shown in FIGS. 2-3. The prosthetic heart valve that incorporates frame 200 may include prosthetic leaflets similar or identical to those described in connection with frame 100.

In use, the prosthetic heart valve incorporating the frame 200 is typically maintained collapsed in a crimped condition over a deflated balloon of a delivery device as it is delivered intravascularly. Because the material forming frame 200 is plastically expandable, it does not require an overlying sheath to maintain it in the collapsed condition. To deploy the prosthetic heart valve, fluid (e.g. saline) may be pumped through the delivery device and into the balloon, forcing the balloon to expand, and thus forcing the prosthetic heart valve that incorporates frame 200 to expand.

As noted above, frames formed of materials that are suitable for balloon-expandable frames, such as stainless steel or cobalt chromium, may have both benefits and drawbacks over other materials (e.g. Nitinol) that are typically used in self-expandable frames. After full deployment and expansion of the prosthetic heart valve that incorporates frame 200, the prosthetic heart valve is maintained in place solely (or nearly solely) by outward radial force from contacting tissue within the native aortic valve. Contrary to Nitinol and similar shape memory materials, materials used in balloon-expandable frames are significantly stiffer and thus, after being forced to expand (e.g. via a balloon) into the native valve annulus, there is no need for additional mechanisms to maintain strong anchoring of the prosthetic heart valve. Because of this, frame 200 may omit outwardly flared sections like those in frame 100, as additional anchoring is not required. One important benefit of this is that, compared to frame 100, frame 200 may be significantly shorter (in the axial direction between the inflow end 202 and outflow end 204). Thus, there is not a significant amount of material of the frame 200 that extends near the coronary arteries. As a result, the frame 200 is far less likely to obstruct blood flow to the coronary arteries, or inhibit future access to the coronary arteries for future interventions.

Although the high stiffness of stainless steel, cobalt chromium and other similar materials used to form balloon-expandable frames may be an advantage because it allows for strong anchoring with minimal height, the high stiffness may also provide certain drawbacks. As noted above in connection with frame 100, the tissue-CAF connection is stressed every time the prosthetic leaflets coapt. This stress is represented by force lines F in FIG. 3. Unlike the CAFs 118 of frame 100, CAFs 218 (and adjacent portions of the frame) will not deflect significantly inwardly when the prosthetic leaflets coapt due to the high stiffness of the material of the frame 200. Without deflection of the CAFs 218, forces applied at the leaflet-CAF connection may not be significantly distributed, meaning that more stress is applied to the soft tissue (or synthetic/fabric) leaflets. This, in turn, may lead to a lower durability of the prosthetic leaflets over time. Stated more simply, the material of balloon-expanding frames may reduce the durability of the “soft” components of the prosthetic heart valve (a drawback) but typically allows for only minimal frame extension into the aorta (a benefit).

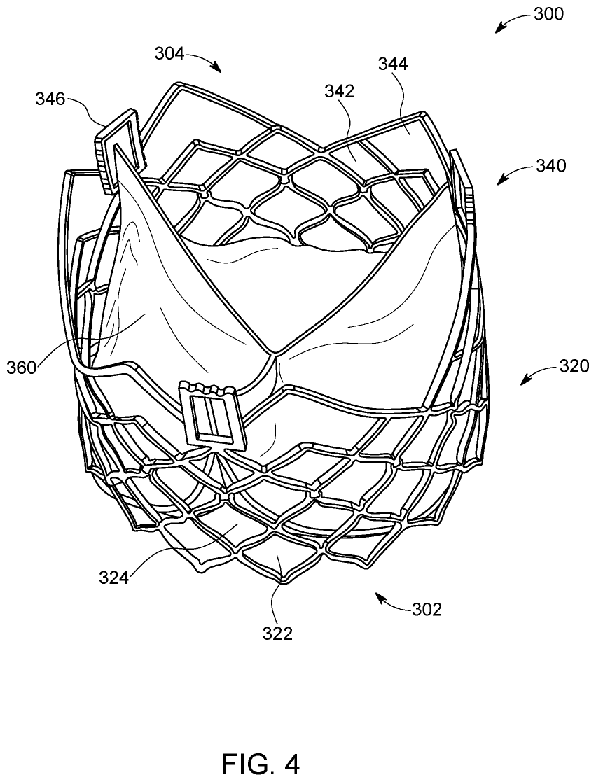

FIG. 4 illustrates a prosthetic heart valve 300 according to an aspect of the disclosure. In the illustrated embodiment, prosthetic heart valve 300 is suited for an aortic valve replacement, but it should be understood that the concepts described herein may be applied to frames for use with other prosthetic heart valves, such as the pulmonary valve. Prosthetic heart valve 300 may extend from an inflow end 302 to an outflow end 304. Overall, when in the expanded condition shown in FIG. 4, prosthetic heart valve 300 may be generally cylindrical, more similar to frame 200 than frame 100 in shape.

Prosthetic heart valve 300 includes a hybrid frame that includes two frame portions. The two frame portions are formed of different materials and, preferably, are not monolithic with each other and thus must be connected after being formed. These two frame portions include a first frame 320 nearer the inflow end 302 of the prosthetic heart valve 300, and a second frame 340 nearer the outflow end 304 of the prosthetic heart valve 300. The first frame 320 is preferably formed from material, such as stainless steel or cobalt chrome, such that the first frame 320 is plastically expandable. The second frame 340 is preferably formed from shape-memory material, such as Nitinol, so that the second frame 340 is self-expanding.

In the illustrated embodiment, the first frame 320 includes two rows of generally diamond-shaped cells, including a first inflow-most row 322 and a second adjacent row 324. It should be understood that the first frame 320 may include a different number of rows of cells, number of cells per row, and shape of the cells than shown. The rows 322 and 324 of first frame 320 may generally correspond to the rows 212, 214 of frame 200.

In the illustrated embodiment, the second frame 340 includes two rows of cells, including a first row of generally diamond-shaped cells 342, and an outflow-most row of irregularly-shaped cells 344. It should be understood that the second frame 340 may include a different number of rows of cells, number of cells per row, and shape of the cells than shown. The rows 342 and 344 of first frame 340 may generally correspond to the rows 214, 220 of frame 200, despite being formed of a different material than frame 200, as described in greater detail below.

As with frame 200, first frame 320 may be formed of a material, such as stainless steel or cobalt chromium, so that the first frame 320 is plastically expandable, for example by inflating a balloon positioned within the first frame 320. Due to the high rigidity of the first frame 320, most or all of the anchoring of prosthetic heart valve 300 may be a result of the radially outward forces that the first frame 320 applies to the native valve (e.g. the native aortic valve annulus). As with frame 100, the second frame 340 may be formed of a material having shape-memory properties, such as Nitinol. The second frame 340 may be shape set, for example via heat treatment, to be biased to a shape similar to that shown in FIG. 4, so that the second frame 340 is self-expandable, from a radially collapsed condition, to the shape shown in FIG. 4.

Importantly, referring still to FIG. 4, the second frame 340 may include a plurality of CAFs 346. In the illustrated embodiment, each CAF 346 is formed integrally with the second frame 340 and has a generally rectangular shape with a rectangular opening. The bottom (inflow end) of each CAF 346 may couple to an outflow apex of a corresponding one of the cells in row 342, and the lateral sides of each CAF 346 may connect to a strut of a respective cell 344, for example near an axial center position of the CAF 346, although other connection points may be suitable. It should be understood that the specific shape of CAF 346 is merely exemplary, and other suitable CAF shapes, types, and/or numbers may be provided, including the style of CAFs shown in FIGS. 1-3, or any other style CAF.

Still referring to FIG. 4, the prosthetic heart valve 300 may include a plurality of prosthetic leaflets 360. In the illustrated embodiment, three leaflets 360 are provided, and as with the prosthetic leaflets described above, prosthetic leaflets 360 may be formed of biological materials (such as tissue), synthetic materials, or combinations thereof. Each adjacent pair of leaflets 360 comes together at a commissure, and that commissure is coupled to the second frame 340 via a respective CAF 346. The commissures may be directly attached to the CAFs 346 via suturing or any other mechanical connection. In other embodiments, one or more intervening components may be used for an indirect connection. For example, one or more layers of fabric (or other materials) may be coupled directly to each CAF 346 so that the layer(s) span the open interior space of the CAF, and the commissures of the leaflets may be directly attached to that layer(s) of material. It should be understood that, in FIG. 4, an intermediate connection layer on CAF 346 is omitted for clarity of the figures. And, although not shown in FIG. 4 for clarity, an internal cuff may be provided interior to one or both frames 320, 340 and/or an exterior cuff may be provided exterior to one or both frames 320, 340. Preferably, at least an interior cuff is provided, and the prosthetic leaflets 360 are attached directly to the interior cuff (and not directly to the stent) along the belly of each leaflet 360 between the CAFs 346. With this particular configuration, each prosthetic leaflet 360 is not attached directly to the first frame 320, and is attached directly to the second frame 340 (if at all), only at the CAFs 346. The prosthetic leaflets 360 are shown in FIG. 4 in the closed or coapted condition. Further, it should be understood that the prosthetic leaflets 360 may be positioned within the hybrid frame along an axial extent that spans both the self-expanding frame 340 and the balloon-expandable frame 320.

Because the first frame 320 provides suitable anchoring force to the entire prosthetic heart valve 300, the prosthetic heart valve 300 does not require additional outward flares like those shown in FIG. 1. In other words, the prosthetic heart valve 300, when expanded, may have the same overall shape and same overall height as frame 200 when expanded. As explained above, this short height is an advantage. However, when the prosthetic leaflets 360 close (as shown in FIG. 4) and the prosthetic leaflets 360 resist back pressure, the CAFs 346 (and/or structure of second frame 340 adjacent to the CAF 346) are capable of significant deflection, due to the material forming the second frame 340. As explained above, this deflection may reduce the stress on, and thus increase the durability of, the “soft” components of the prosthetic heart valve 300, including the prosthetic leaflets 360 and other fabric or tissue materials (such as an inner cuff) disposed on the hybrid frame near the CAFs 346. Thus, as should be understood, the hybrid frame used by prosthetic heart valve 300 provides certain benefits of balloon-expandable frames and self-expandable frames, while avoiding certain drawbacks of both balloon-expandable frames and self-expandable frames.

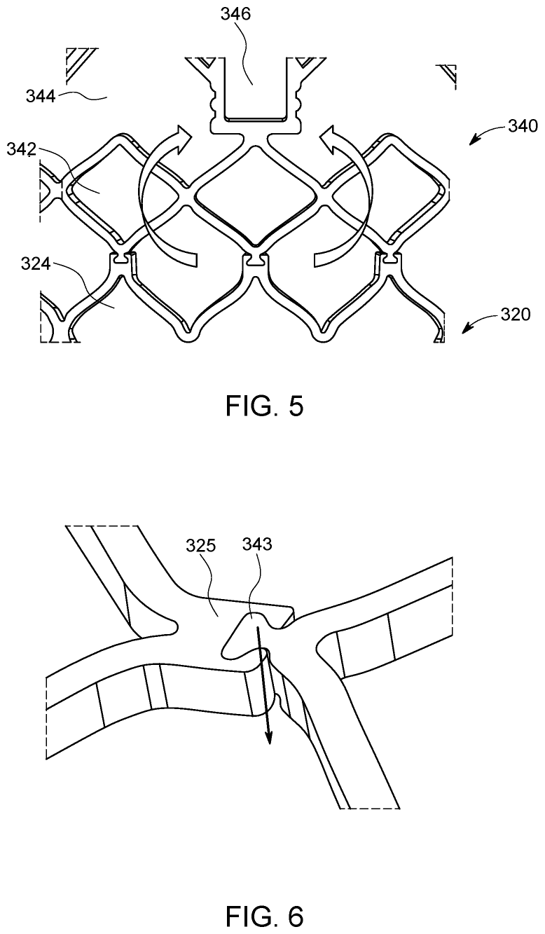

In some embodiments, the hybrid frame is formed as a monolithic structure, such as by additive manufacturing. If the frame is formed by additive manufacturing, it may be formed with a transition section in which the ratio of cobalt chromium (or other plastically expandable metal) to nitinol (or other shape memory metal) gradually transitions from 100:0 to 0:100. However, in other embodiments (including the embodiment of FIGS. 4-6), the frames 320, 340 may be formed separately and then connected together after initial manufacturing. The connection mechanism shown between frames 320, 340 in FIGS. 4-5 aims to, inter alia, reduce relative motion between the two frames for maximum structural integrity between the two frames.

One potential option for this connection is to wedge the nitinol frame inside the cobalt chromium frame to prevent inward motion of the nitinol frame during valve closing (see FIG. 5). For example, referring to FIG. 6, the second frame 340 may include a plurality of tabs 343, for example extending from inflow apices of cells 342, and first frame 320 may include a plurality of corresponding extensions 325 extending from outflow apices of cells 324, the extensions 325 forming recesses that are generally complementary to the shape of the tabs 343. In the illustrated embodiment, the tabs 343 are received within the recesses of the extensions 325 in a general dove-tail configuration to prevent the tabs 343 from axially pulling out from the extensions 325. However, the tabs 343 and/or the recesses of the extensions 325 may have a slightly tapering width (width being measured in the circumferential direction) along the thickness of the component to form a general wedge. In the example of FIG. 6, the tabs 343 may be slightly wider (in the circumferential direction) on the abluminal-facing side compared to the luminal-facing side. Thus, the relatively wider abluminal surface of the tabs 343 helps prevent the tabs 343 from pulling radially inwardly as the prosthetic leaflets 360 close and tend to pull the second stent 340 radially inwardly.

There are other configurations of the connection than that shown in FIGS. 5-6. In one example, the wedge direction described above may be reversed to intentionally allow for additional inward motion of the second frame 340 during closing of leaflets 360 to provide additional deflection, but in those cases, additional connection mechanisms may be desirable to ensure the second frame 340 does not fully disconnected from the first frame 320. In another example, the tabs 343 may include an additional ridge that snaps or otherwise locks within a complementary groove within the recess of the extension 325 (or vice versa) to provide enhanced interlocking between the two frames 320, 340.

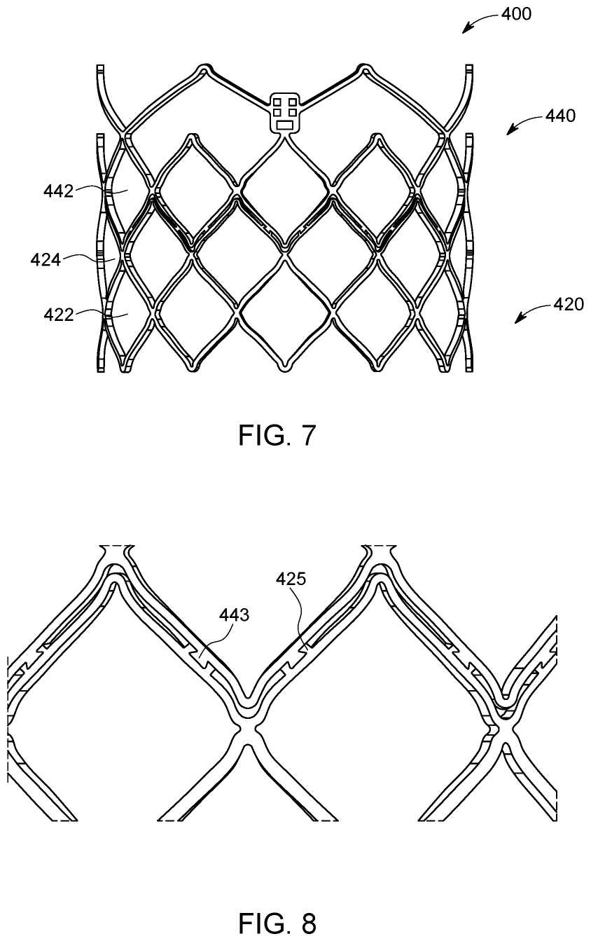

FIG. 7 illustrates a prosthetic heart valve 400 (with prosthetic leaflets and inner and/or outer cuffs omitted from the figure) that generally corresponds to prosthetic heart valve 300, with a first balloon-expandable frame 420 that corresponds to first frame 320, and a second self-expanding frame 440 that corresponds to second frame 440. The main difference between prosthetic heart valves 300 and 400 is the specific mechanism and configuration by which the two different frames connect. For example, in prosthetic heart valve 300, the two frames 320, 340 engage each other at corresponding apices of diamond-shaped cells (e.g. outflow apices of cells 324 engage with inflow apices of cells 342). In prosthetic heart valve 400, the first frame 420 includes two rows 422, 424 of diamond-shaped cells, and the second frame 440 may include a single row 442 of diamond-shaped cells. However, instead of the outflow apices of cells 424 engaging the inflow apices of cells 442, the outflow half of each cell 424 is nested between the inflow half of two circumferentially adjacent cells 442. With this configuration, best shown in FIG. 8, there is more strut structure available for engagement between the two frames, instead of engagement only occurring at the apices of corresponding cells. In other words, each cell 424 may be coupled at two or more locations to cells 442. The specific mechanism for coupling may be similar to those described above, for example including wedged tabs 443 received within recesses of extensions 425 to form a dovetail connection. In the illustrated embodiment, each strut on the outflow half of each cell 424 includes one extension 425, and each strut on the inflow half of each cell 442 includes a corresponding tab 443. Compared to prosthetic heart valve 300, prosthetic heart valve 400 has double the number of connections which may provide enhanced stability of connection. However, it should be understood that even more connections than shown in FIG. 8 may be provided due to the fact that the confronting struts of cells 424 and 442 are in close proximity along their entire lengths.

In use, the prosthetic heart valves 300, 400 may be collapsed prior to delivery into the patient. Because the first frames 320, 420 are significantly stiffer than the second frames 340, 440, the second frames 340, 440 will be mostly or entirely prevented from self-expanding as long as the first frame 320, 420 remains collapsed. Thus, in some embodiments, the entire prosthetic heart valve 300, 400 may be crimped over a balloon of a delivery device without the need for any overlying sheath. In other embodiments, it may be desirable to have an overlying sheath to cover all of the prosthetic heart valve 300, 400, to cover only the second frame 340, 440, or to cover only the terminal outflow end of the second frame 340, 440. If such an overlying sheath is used, the overlying sheath may only need to cover the terminal outflow end of the second frame 340, 440 to prevent that terminal end from flaring outward due to the self-expanding properties of the second frame 340, 440. Otherwise, the delivery procedure may follow that of a standard balloon expandable valve. Once the prosthetic heart valve 300, 400 is in the desired target position (e.g. at the native aortic valve), the underlying balloon may be inflated to force the first frame 320, 420 to expand. If an overlying sheath is used, it would be removed prior to the expansion of the balloon. The balloon force will cause the first frame 320, 420 to expand, with the second frame 340, 440 following with expansion due to the balloon force and/or due to the self-expanding properties of the second frame 340, 440. After deployment of the prosthetic heart valve 300, 400, the balloon may be deflated and the delivery device withdrawn from the patient.

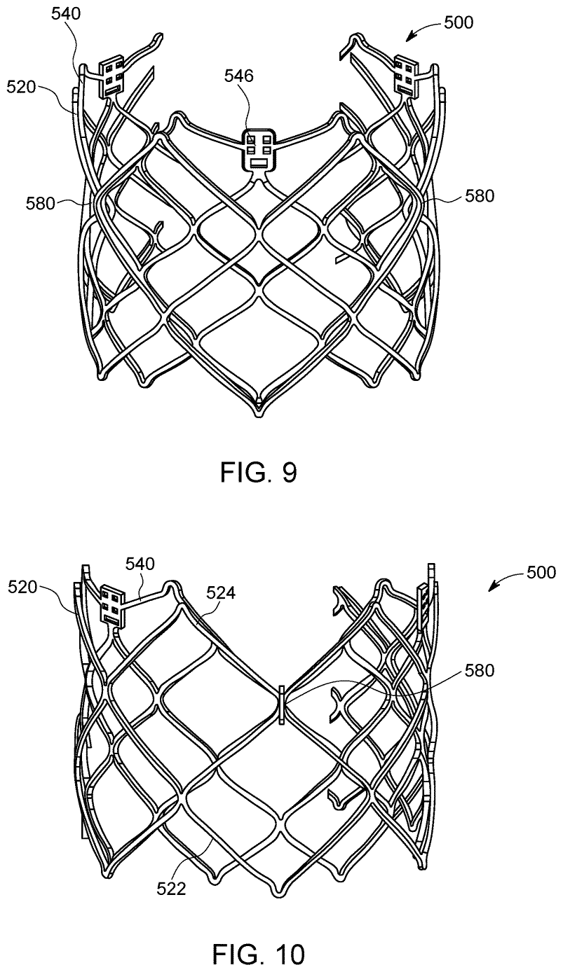

FIGS. 9-10 illustrate a prosthetic heart valve 500, in an expanded condition, according to another aspect of the disclosure. The prosthetic heart valve 500 of FIGS. 9-10 is illustrated with the interior prosthetic leaflets and an internal and/or outer cuff omitted from the view. The view of FIG. 9 is rotated about 60 degrees relative to the view of FIG. 10. Like prosthetic heart valve 300, 400, prosthetic heart valve 500 includes both a first balloon-expandable frame 520 and a second self-expanding frame 540. However, instead of stacking the two frames axially, the two frames are positioned in a radially overlapping manner in prosthetic heart valve 500. In particular, the self-expanding (e.g. nitinol) second frame 540 is positioned radially inside the plastically-expandable (e.g. cobalt chromium) first frame 520. In the illustrated embodiment, both frames 520, 540 have about the same height (in the axial direction) in the expanded condition of the hybrid frame.

Generally, the outer balloon-expandable frame 520 serves the purpose of anchoring the prosthetic heart valve in the anatomy (e.g. within the native aortic valve annulus). The inner self-expanding frame 540 serves the purpose of supporting the prosthetic leaflets, for example at CAFs 546. In this configuration, the inner frame 540 can be optimized for prosthetic valve performance, and the outer frame 520 can be optimized for anchoring. For example, the outer frame 520 includes diamond-shaped cells, but instead of smaller diamond-shaped cells, frame 520 includes large diamond-shaped cells 522, 524 with low opening angles. In the illustrated embodiment, the entire outer frame 520 is formed by only two axial row of diamond-shaped cells 522, 524, with a total of six cells in each row. This atypically large cell size may reduce the effects of axial lengthening of the outer frame 520 during balloon deployment. With large diamonds, but wide strut widths, optimized anchoring of the prosthetic valve 500 (via the outer frame 520) may be achieved with less overall structure. Such large, open structures as the diamond-shaped cell configuration of the outer frame 520 of FIGS. 9-10 are prohibitive in a single frame design as they provide very little support structure for placement of features for the “soft” components of the prosthetic valve, such as prosthetic leaflets and inner and/or outer cuffs. However, since the inner frame 540 does not need to provide anchoring, its structure can be optimized for leaflet/valve performance. For example, the inner frame 540 may include diamond-shaped cells, but in a greater number (and smaller size per cell) compared to the outer frame 520. As with the embodiments described above, the material forming the inner self-expandable frame provide enhanced deflection. It should be understood that, although the term “self-expandable” is used in connection with frame 540, it may not actually be self-expandable in the sense that the forces of the inner frame 540 that tend to cause the inner frame 540 to “want” to self-expand are too small to overcome, alone, the very stiff construction of the outer frame 520.

One downside of a purely balloon-expandable valve is expansion uniformity. For example, in a prosthetic heart valve that is supported only by a balloon-expandable frame, the frame must be collapsed by compression forces to “crimp” the valve over a balloon for delivery, and the balloon must expand outwardly to force the frame to expand during deployment. This external application of forces (either to crimp or to expand) may lead to non-uniformity of the valve if the forces are not perfectly and symmetrically applied. For example, when the balloon is inflated to force the valve to expand, if the CAFs do not expand in a completely uniform way, the leaflets may sub-optimally close, leading to poor valve performance and leaflet durability. With the embodiment of FIGS. 9-10, however, as the balloon expands within the pair of frames 520, 540, the tendency of the self-expanding frame 540 to want to return to its pre-set shape may help “guide” the expansion of the balloon expandable frame 520, even though the majority of the forces causing the balloon-expandable frame 520 are from the balloon. In other words, the self-expanding frame 540 may provide relatively small forces to the outer frame 520, but those forces may be enough to help the outer frame 520 expand in a more uniform manner. It should be understood that this benefit may work in reverse as well, so that the self-expanding frame 540 may help the outer frame 520 achieve a more uniform shape during crimping of the prosthetic heart valve 500 onto a balloon for delivery. It should also be understood that no overlying sheath is necessary when delivering and deploying prosthetic heart valve 500, as the self-expansion forces of the inner frame 540 are not large enough to cause the prosthetic heart valve 500 to prematurely expand prior to balloon inflation.

The connection between the first outer frame 520 and the second inner frame 540 may be provided in any suitable fashion. One such method is shown in FIG. 9-10, where the connection is placed opposite of the CAFs 546 to maximize the deflection of the inner frame 540 at the CAF. The connections 580 may be sutures, clips, clamps, or any other suitable mechanical connection that couples the inner frame 540 to the outer frame 520, for example at three locations around the circumference of the prosthetic heart valve. The connection location between the two frames 520, 540 may be very stiff due to the outer frame 520 being formed as a plastically expandable frame. In some embodiments, it may be preferable to include fabric, tissue or other “soft” material at points where the outer frame 520 may contact the inner frame 540, which may prevent metal-on-metal contact which may be desirable. These points may include, but are not limited to, the connections 580. It should also be noted that the foreshortening factors are different between the diamond-cells of the inner frame 540 compared to those of the outer frame 520. In some embodiments, the connections 580 are only positioned at three locations, with this positioning helping to allow the hybrid frame to expand, with the inner and outer frames expanding at different foreshortening rates, without the two frames binding up on each other.

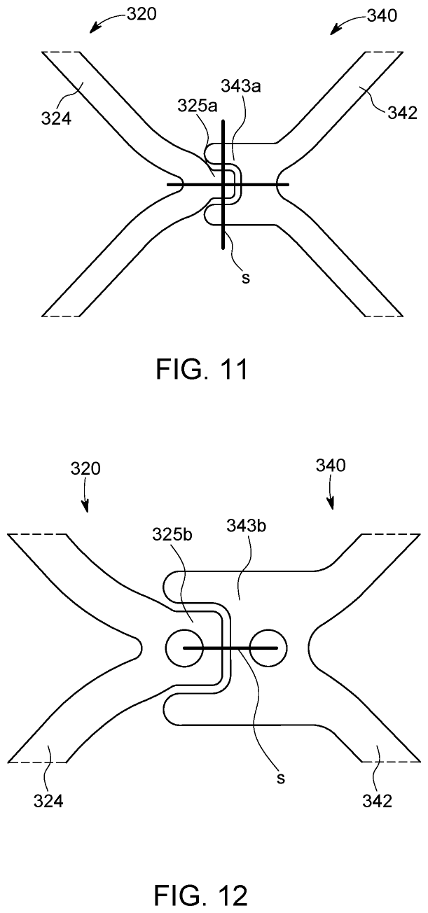

As noted above, it may generally be desirable to avoid metal-on-metal contact between the first frames 320, 420, 520 and their corresponding second frames 340, 440, 540. FIGS. 11-14 illustrate various alternative connection configurations for connecting the first frame to the second frame, although these embodiments primarily apply to the hybrid frame design type shown in connection with prosthetic heart valves 300, 400. For ease of reference, FIGS. 11-14 are described in labeled below in the context of prosthetic heart valve 300.

In FIGS. 11-14, a single cell apex of the first, balloon-expandable frame 320 is shown at a single connection point to a single cell apex of the second, self-expandable frame 340. In the views of FIGS. 11-14, the outflow side of the valve is to the right of the figure, and the inflow side of the valve is to the left in the figure, and the views focus on the connection between an outflow apex of a cell 324 of the balloon-expandable frame 320 to an inflow apex of a cell 342 of the self-expandable frame 340.

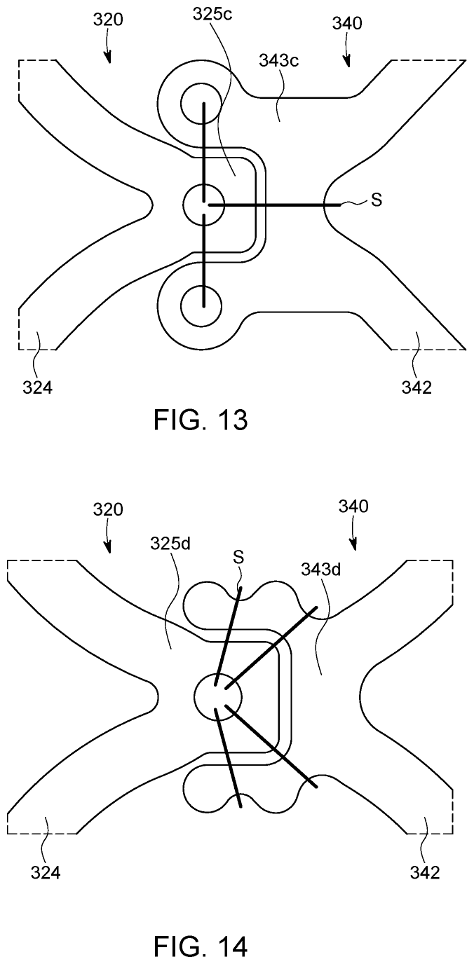

FIG. 11 shows a connection method in which the apex of cell 324 includes a protrusion 325a that is received within a recess of an extension 343a at the apex of cell 342. The protrusion 325a and recess of the extension 343a may be sized so that a small gap remains even when the protrusion 325a is received within the recess of the extensions 343a. A non-metal buffer, such as one or more layers of fabric and/or tissue, may be positioned within the gap so that the metal of the protrusion does not directly contact the metal of the extension 343a forming the recess. In FIG. 11, two suture wraps S are shown in a generally cross-cross pattern with one suture wrap received within the valley of the corresponding apices of cells 324, 342, with another suture wrap S around the extension 343a where the protrusion 325a is received. FIG. 12 is generally similar to FIG. 11, except that the extension 343b may include an aperture and the protrusion 325b may include an aperture, so that the suture wrap S can pass through the two apertures to connect the two components more easily and more securely. The configuration of FIG. 13 is generally similar to FIG. 12, except that the extension 343c includes apertures in the two sides on either side of the recess formed by the extension 343c, so that the two apertures in the extension 343c generally align with the aperture in the protrusion 325c. With this configuration, three suture wraps S may be used, with each suture wrap passing through the aperture in the protrusion 325c. One suture wrap S may also pass through one of the apertures in extension 343c, another suture wrap S may also pass through the other one of the apertures in extension 343c, and the third suture wrap S may wrap around the valley defined by the apex of cell 342. The configuration of FIG. 14 is generally similar to that of FIG. 13, but instead of apertures in the extension 343d, the extension may include a plurality of bump-outs or ridges that form local valleys between adjacent ridges or between a ridge and a strut of cell 342. With this configuration suture wraps S may pass through the aperture in protrusion 325d and wrap around a local valley defined between adjacent ridges or between a ridge and a strut of cell 342, with the local valley helping maintain the suture wrap S in the desired position without slipping. It should be understood that all of the configurations of FIGS. 11-14 may include the gap described in connection with FIG. 11 to receive a buffer, such as fabric or tissue, to prevent or minimize metal-on-metal contact.

It should also be understood, in reference to FIG. 6 and FIGS. 11-14, that the positions of the protrusions may be swapped with the positions of the recess-forming extensions. Further, while specific configurations of suture wrapping, along with features to assist with such suture wrapping, are shown in FIGS. 11-14, these configurations should be understood to illustrate examples and not limit the way in which the first frame 320 may connect to the second frame 340.

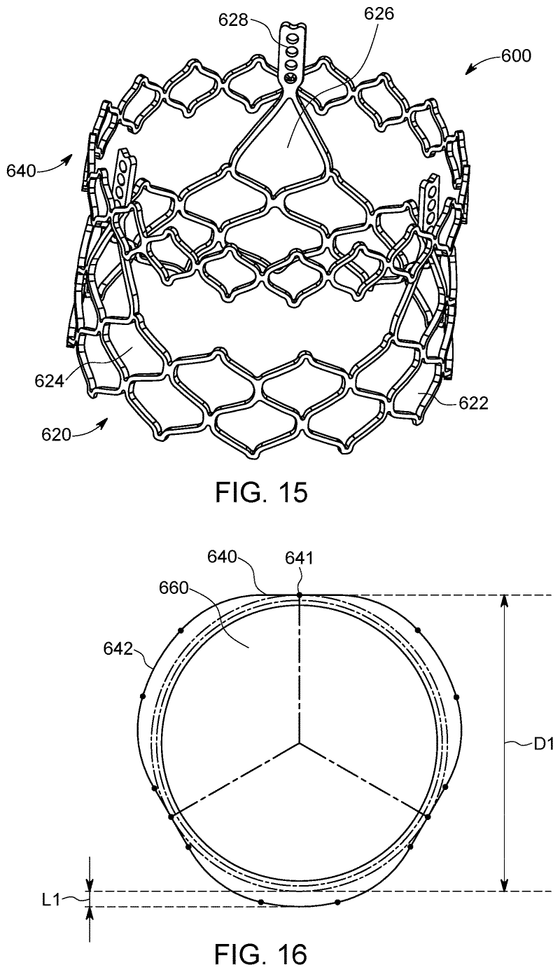

FIG. 15 illustrates a prosthetic heart valve 600 that has features in common with the other prosthetic heart valves described herein, namely the use of a hybrid frame that includes a main balloon expandable frame 620 and a self-expandable frame 640. As with other embodiments, the “soft” materials, such as the prosthetic leaflets and inner and/or outer cuffs, are omitted from FIG. 15. In FIG. 15, the main frame 620 is a relatively rigid balloon-expandable frame, for example formed of cobalt chromium, stainless steel, etc. As with the other embodiments herein, the balloon-expandable frame 620 primarily functions to anchor the prosthetic heart valve 600 within the native valve annulus. However, in this embodiment the balloon-expandable frame 620 also supports the leaflets at CAFs 628. In the illustrated embodiment, the balloon-expandable frame 620 includes two rows of generally diamond-shaped cells which provide the main anchoring force against the annulus when forced to plastically expand, for example via inflation of a balloon over which the prosthetic heart valve 600 is crimped. However, unique to this embodiment is that the balloon-expandable frame 620 also includes selected cells 626 extending away from cells 624, with the selected cells 626 terminating in a CAF 628, which in the illustrated embodiment is generally rectangular with a plurality of holes formed therein (e.g. in a single column) for assisting with suturing the prosthetic leaflets to the CAFs 628. It should be noted that the number of selected cells 626 preferably corresponds to the number of CAFs 628, so that if there are two or three CAFs 628 (because there are two or three prosthetic leaflets), preferably only two or three selected cells 626 are correspondingly included. With this configuration, the selected cells 626 are not attached to additional cells of the balloon-expandable frame 620 in the circumferential direction, such that the selected cells 626 have relatively littler structural support compared to cells 622, 624. The effect of this is that the selected cells 626, and particularly the CAFs 628 extending therefrom, have a cantilevered configuration in which the CAFs 628 are capable of significant deflection, despite the fact that the material forming the selected cells 626 and CAFs 628 is a highly rigid material like cobalt chromium or stainless steel. And even though particular shapes and sizes are shown for selected cells 626 and CAFs 628 in FIG. 15, it should be understood the geometry, length of extension, and other properties may be modified to achieve a larger or smaller amount of cantilevering and/or deflection of the CAFs 628.

Still referring to FIG. 15, it should be understood that this hybrid frame solves the commissure deflection problem at least in part via the geometry and positioning of the selected cells 626 and CAFs 628. However, during deployment, as the underlying balloon forces the first frame 620 to expand, the fact that the selected cells 626 and CAFs 628 are relatively unsupported may lead to asymmetric expansion (and eventual positioning) of the CAFs 628. As noted above, if the positioning of the CAFs 628 after deployment is not optimal, leaflet closing dynamics may be negatively affected, possibly leading to blood leaking through the valve and/or low durability of the leaflets. Depending on the amount of cantilevering of the CAFs 628, the CAFs 628 may also be at risk of too much deflection. In order to solve both of these potential problems, the second frame 640 is provided. The second frame 640 may be in the form of a “commissure ring” as generally described in U.S. Provisional Patent Application No. 63/384,521 filed Nov. 21, 2022 and titled “Transcatheter Prosthetic Atrioventricular Valve with Stiffening Structure,” the disclosure of which is hereby incorporated by reference herein.

In the illustrated embodiment of FIG. 15, the second frame or commissure ring 640 is formed as self-expanding frame that includes a single row of generally diamond-shaped cells, with the commissure ring 640 being generally circular in the absence of applied forces. The commissure ring 640 may be positioned exterior to the first frame 620 at or near the CAFs 628, and preferably is connected directly to the CAFs 628 (with or without an intermediate buffer material to reduce or eliminate metal-on-metal contact), for example via suturing through one or more holes in the CAFs 628. Notably, when the prosthetic heart valve 600 is expanded as shown in FIG. 15, there is a significant gap space between the cells 624 and the commissure ring 640 between the selected cells 626. This gap space preferably remains uncovered by cuffs or other “soft” components to promote open flow of blood to the coronary arteries if prosthetic heart valve 600 is used to replace a malfunctioning aortic valve.

In use, the prosthetic heart valve 600 may be crimped over a balloon of a delivery device for delivery. The commissure ring 640 may remain uncovered by any overlying sheath (if desired) and the stiffness of the collapsed frame 620 will maintain the commissure ring 640 in the collapsed state during delivery. When the prosthetic heart valve 600 reaches the valve to be replaced (e.g. via a transfemoral delivery to the aortic valve), the balloon may be inflated to force the frame 620 to expand and anchor into the native annulus. Importantly, even if the balloon unevenly or asymmetrically applies opening force to the region of the CAFs 628 of the balloon-expandable frame 620, the commissure ring 640 will nonetheless naturally revert to its set-shape, helping to pull the CAFs 628 into the desired symmetric/uniform positioning. Further, during operation when the prosthetic leaflets close, the commissure ring 640 may assist in preventing over-deflection of the CAFs 628 if such over-deflection is a risk. Still further, if the commissure ring 628 were absent, all of the forces from the deflection of the CAFs 628 during leaflet closing would be concentrated at or near the connection of the struts of the selected cells 626 to cells 624. In the presence of the commissure ring 640, those forces may be more significantly disperses through the structure of the commissure ring 640. The commissure ring 640, being formed of a self-expanding material that is flexible, may more readily absorb those forces in a way that is not likely to cause any fatigue or other structural damage to the frame of the prosthetic heart valve 600.

Although commissure ring 640 is shown as having a circular shape in the expanded (or shape-set) condition, other options are possible. For example, if the commissure ring 640 is circular, there may be a risk that the prosthetic leaflets, upon opening, contact the metal of the commissure ring 640, which could lead to premature deterioration of the prosthetic leaflets. In another example, the commissure ring 640 may be provided as having a lobed shape, as shown in FIG. 16. For example, FIG. 16 shows a schematic of commissure support 640 which, in its expanded condition, has a plurality of lobes. For example, in the given example, the prosthetic heart valve includes three prosthetic leaflets 660, and thus the commissure support 640 is formed (e.g., via shape setting) to have three lobes. FIG. 16 shows that an outer diameter of the frame 620 at the position of the outer surface of the CAFs 628 of the frame 620 has a value of D1. Because the leaflets 660 are coupled to the CAFs 628, the leaflets 660 at these locations do not change position (or at least not significantly) during opening and closing of the prosthetic leaflets 660. Thus, at these locations the commissure ring 640 has a minimum extent 641 that is in contact with the outer surface of the CAFs 628. However, as the prosthetic leaflets 660 open, the middle of the free edges is typically the portion that may have the greatest movement outwardly. In order to account for the possibly large movement, the commissure ring 640 increases in terms of radial extent from the longitudinal center of the prosthetic heart valve 600 to a maximum extent 642 that is positioned about midway along the perimeter of the commissure support 640 between two adjacent minimum extents 641. At this maximum extent 642, the distance between the outer surface of the frame 620 and the commissure support 640 may have a length L1. Although the length L1 may be adjusted as desired, in one example the length L1 is about 1.5 mm. In other words, the middle of the free edges of the prosthetic leaflets 660 may elongate up to about 1.5 mm beyond the diameter D1 without interference from the commissure ring 640. In a prosthetic heart valve 600 with three leaflets 660, the illustrated commissure ring 640 in FIG. 16 would have three minimum extents 641 positioned about 120 degrees apart (aligned with the CAFs 628), and three maximum extents 642 positioned about 120 degrees apart (generally aligned with the middle of the free edges of the prosthetic leaflets 660) with the maximum extents 642 being offset by about 60 degrees from the minimum extents 641. The particular shape of the transition between the minimum extents 641 and maximum extents 642 may vary, but preferably create a lobe shape similar to that shown in FIG. 16 to provide for a large amount of extra space for movement of the prosthetic leaflets 660. It should be understood that this lobed-shaped commissure ring 640 is typically not achievable (or at least not easily achievable) with plastically-expandable materials because plastically-expandable frame are typically expanded via balloons that have generally cylindrical shapes, which is the shape that the balloon-expandable shape typically takes upon expansion.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve, comprising:
a balloon-expandable frame formed of a plastically-expandable material;
a self-expanding frame formed of a self-expanding material, the self-expanding frame including a plurality of commissure attachment features; and
a plurality of prosthetic leaflets, wherein pairs of adjacent prosthetic leaflets form commissures, each of the commissures being coupled to a corresponding one of the plurality of commissure attachment features;
wherein the balloon-expandable frame is coupled to the self-expanding frame to form a hybrid frame, the hybrid frame including an inflow end and an outflow end, the balloon-expandable frame being positioned at the inflow end of the hybrid frame, and the self-expanding frame being positioned at the outflow end of the hybrid frame.

2. The prosthetic heart valve of claim 1, wherein the plurality of prosthetic leaflets are positioned within the hybrid frame along an axial extent that spans both the self-expanding frame and the balloon-expandable frame.

3. The prosthetic heart valve of claim 1, wherein the balloon-expandable frame includes a first inflow-most row of cells, and a second row of cells positioned downstream of the first row of cells, and the self-expanding frame includes a third row of cells.

4. The prosthetic heart valve of claim 3, wherein the balloon-expandable frame is coupled to the self-expanding frame via connections between the second row of cells and the third row of cells.

5. The prosthetic heart valve of claim 4, wherein the connections each include one of a protrusion or recess positioned at an outflow apex of a cell in the second row of cells, and the other of a protrusion or recess positioned at an inflow apex of a cell in the third row of cells, the protrusion being received within the recess.

6. The prosthetic heart valve of claim 5, wherein a buffer material is positioned between each corresponding protrusion and recess to prevent metal-on-metal contact at the connections.

7. The prosthetic heart valve of claim 5, wherein each protrusion or recess is wedged in a thickness direction of the hybrid frame to prevent the self-expanding frame from sliding radially inwardly with respect to the balloon-expandable frame at the connections.

8. The prosthetic heart valve of claim 5, wherein the protrusion and the recess form a dovetail connection.

9. The prosthetic heart valve of claim 5, further comprising one or more sutures wrapping around each of the connections to secure the protrusion within the recess.

10. The prosthetic heart valve of claim 4, wherein an outflow apex of a cell in the second row of cells is positioned between two circumferentially adjacent inflow apices of two adjacent cells in the third row of cells.

11. The prosthetic heart valve of claim 10, wherein the connections each include one of a protrusion or recess positioned on a strut of the cell in the second row of cells, and the other of a protrusion or recess positioned on a strut of one of the two circumferentially adjacent cells in the third row of cells, the protrusion being received within the recess.

12. A prosthetic heart valve, comprising:
a balloon-expandable frame formed of a plastically-expandable material;
a self-expanding frame formed of a self-expanding material, the self-expanding frame including a plurality of commissure attachment features; and
a plurality of prosthetic leaflets, wherein pairs of adjacent prosthetic leaflets form commissures, each of the commissures being coupled to a corresponding one of the plurality of commissure attachment features;
wherein the self-expandable frame is coupled to, and positioned radially within, the balloon-expandable frame to form a hybrid frame.

13. The prosthetic heart valve of claim 12, wherein in an expanded condition of the hybrid frame, the balloon-expandable frame has a first axial height and the self-expanding frame has a second axial height about equal to the first axial height.

14. The prosthetic heart valve of claim 13, wherein the balloon-expandable frame includes a plurality of circumferential rows of diamond-shaped cells, and the self-expanding frame includes a plurality of rows of diamond-shaped cells.

15. The prosthetic heart valve of claim 14, wherein the balloon-expandable frame includes fewer circumferential rows of diamond-shaped cells than the self-expanding frame.

16. The prosthetic heart valve of claim 14, wherein each diamond-shaped cell of the balloon-expandable frame defines a first area that is larger than an area of any diamond-shaped cell of the self-expanding frame.

17. A prosthetic heart valve, comprising:
a balloon-expandable frame formed of a plastically-expandable material, the balloon-expandable frame including a plurality of commissure attachment features;
a self-expanding frame formed of a self-expanding material; and
a plurality of prosthetic leaflets, wherein pairs of adjacent prosthetic leaflets form commissures, each of the commissures being coupled to a corresponding one of the plurality of commissure attachment features;
wherein the self-expanding frame is coupled to the plurality of commissure attachment features to form a hybrid frame, the self-expanding frame being positioned radially outward of the balloon-expandable frame at an axial height that aligns with the plurality of commissure attachment features.

18. The prosthetic heart valve of claim 17, wherein each of the plurality of commissure attachment features is cantilevered with respect to the balloon-expandable frame.

19. The prosthetic heart valve of claim 18, wherein the balloon-expandable frame includes two circumferential rows of diamond-shaped cells adjacent to an inflow end of the hybrid frame, and the self-expanding frame includes one circumferential row of diamond-shaped cells adjacent to an outflow end of the hybrid frame.

20. The prosthetic heart valve of claim 19, wherein in the absence of applied forces, the self-expanding frame is either:
(i) circular; or
(ii) lobed, with one lobe positioned circumferentially between each pair of circumferentially adjacent commissure attachment features, each lobe extending a first distance from a radial center of the self-expanding frame, the self-expanding frame having connection locations where the self-expanding frame is coupled to the plurality of commissure attachment features, each connection location extending a second radial distance from the radial center of the self-expanding frame, the first distance being greater than the second distance.
